(19) United States
(12) Patent Application Publication
(10) Pub. No.: US 2004/0039317 A1
(43) Pub. Date: Feb. 26, 2004

(54) Separable compression sleeve with barrier protection device and reusable coupler

(76) Inventors: Sean J. Souney, Monrovia, CA (US); Rick W. Roeder, Monrovia, CA (US)

Correspondence Address: Sean J. Souney, 730 E. Los Angeles Ave, Monrovia, CA, 91016, US

(21) Appl. No.: 10/226,382

(22) Filed: Aug. 23, 2002

(51) Int. Cl. A61F 5/00

(52) U.S. Cl. 602/13

(57) ABSTRACT

A separable compression sleeve where the fluid inflating portion commonly referred to as bladder, tubing and connector (26) can be separated from the garment or sleeve portion (30) that wraps around the limb or desired substrate to be compressed. The bladder, tubing and connector (26) can be mechanically barrier protected so it can be reused without chemical reprocessing by using a protective flexible covering (32) (barrier), a unique outer or connector coupler shell 10, a membrane barrier (valve) (18), a reusable coupler (12), disposable coupler connector (24) and an easy open heat shrink connection (20) that allows for quick dissassembly of the barrier so the bladder tubing and connector (26) can be reused.

### FEDERALLY SPONSORED RESEARCH

Not Applicable

### SEQUENCE LISTING OR PROGRAM

Not Applicable

### BACKGROUND-FIELD OF INVENTION

This invention relates to a separable compression sleeve with a barrier protection device with a reusable coupler that provides protection from contamination so that the fluid inflation device (bladder, tubing and coupler) can be reused without the possibility of cross contamination from the contaminant to the fluid inflation device and the patient contacting wrap portion can be separated from the compression sleeve for disposal or reprocessing.

### BACKGROUND-DESCRIPTION OF PRIOR ART

Prior art reveals numerous designs in the field of pneumatic compression products that are either one time use or if for multiple use are subjected to sterilization of the entire unit. Also, connectors are utilized at the ends of these devices that can be easily contaminated and do not allow for reuse without a decontamination process or sterilization process. When these current device connectors or couplers are placed at the end of a fluid inflatable device to allow it to be reused without being subjected to a cleaning process, they all suffer from major disadvantages.

(a) Currently, the devices of connection face the outside and inevitably contact the outside. Even in current needle-less type systems there is a failure rate on all systems as they are contacted and penetrated from the outside.

(b) Fluid inflation devices that are essentially compression sleeves that secure around a substrate or limb and inflate a bladder that circumferentially compresses said limb or substrate are divided into two types that are basically handled in two ways. Devices that are for one time use typically are disposed of after use so the issue of designing the device for reuse does not become an issue. Devices that are for multiple use are designed to be reprocessed. Reprocessors that reprocess these devices or single use devices must expose them to some sort or reprocessing procedure which has shortcomings with each method of reprocess. Sterilization of the devices by a sterililant gas results in toxic residues on polymer products (which most of these devices are) or degradation when sterilized with gamma sterilization. Decontamination and cold sterilization results in chemicals that are difficult to completely remove in a device that is attempting to be reused again on a patient especially from the patient contacting wrap portion which is often a fabric.

(c) Sealing a device off from contamination with a safety seal type product that allows reopening through the use of a pull tab requires the plastic seal to be free of holes that would allow contamination. Prior art includes only two types of seals, one where there is a complete seal but no pull tab to easily remove the safety seal after use and one where there is a safety pull tab but the sealed surface is perforated along the length of the device (holes) to allow the pull tab to easily fuinction (tear off) by tearing along the line of perforation.

(d) Barrier membranes block fluid. Prior art has no designs that in ambient air at atmospheric pressure a barrier will function to prevent liquid, tactile or aerosolized contact contamination, yet when connected allow the inflatable fluid devices' pressures to open the membrane.

(e) Current fluid compression devices like blood pressure devices and intermittent pneumatic compression devices are either single use or built heavier for reprocessing do not allow for a protective cover to be built around the product that can be torn away from the product and eliminate the need for chemical reprocessing that would affect deleteriously either the cover or wrap that secures the bladder around the patient/device or the bladder, tube, and connector.

Objects and Advantages

Accordingly, besides the objects and advantages of the barrier protection device for fluid inflation devices with reusable coupler, several objects and advantages of the present invention are:

(a) To allow for the reuse without the need of reprocessing of currently disposable fluid inflation devices such as external intermittent compression devices, blood pressure or tourniquet cuffs with sterilization or chemical treatments.

(b) To allow for the reuse of fluid couplers or connectors without the need for the sterilization or chemical reprocessing of couplers on devices that inflate as they are difficult to reprocess

(c) To provide an easy to use and easy to disassemble safety seal that attaches the barrier around the bladder, tube and reusable coupler.

(d) To provide a unique diaphragm membrane (valve) which can be activated mechanically by either a male spear on one of the coupling or connecting products to open it or merely by design of the living hinge to open with the normal operational fluid movement of the device, yet close off to contamination when the coupler is separated from its fluid pressure source.

(e) To provide a membrane which by design can alter the flow required for the specific device yet still maintain protection from contamination when not in use

(f) To provide a process whereby a separate compression sleeve with a barrier protected inflation device, the unique reusable coupler design, the unique membrane for fluid transfer, and the safety seal which holds all these components together can be separated so that the bladder, tube and coupler can be reused due to the mechanical barrier protection. The barrier can be removed from around the bladder, tube and reusable coupler and then replaced to allow multiple uses and prevent the need for disposal or for chemical reprocessing methods and procedures on the bladder, tubing and reusable coupler.

### SUMMARY

In accordance with the present invention a separable compression sleeve with a barrier protection device around the inflation bladder, tube and connector for fluid inflation devices with a coupler comprises a barrier which consists of a thin flexible covering that can be sealed around shapes of the fluid inflation device(bladder), a reusable coupler with a breakaway disposable portion that contains (trapped) in it a unique membrane that allows for the flow of the fluid when connected and prevents contamination when disconnected and a unique safety seal that connects the previously mentioned components and is easily removable in an aseptic technique so that the barrier, a new disposable end to the connector coupler and a new safety seal can be easily replaced over the device to allow it to be reused over and over without the need for chemical reprocessing procedures to be applied to the fluid inflatable device or the reusable coupler.

### DRAWINGS

### Drawing Figures

In the drawings, closely related figures have the same number but different alphabetic suffixes.

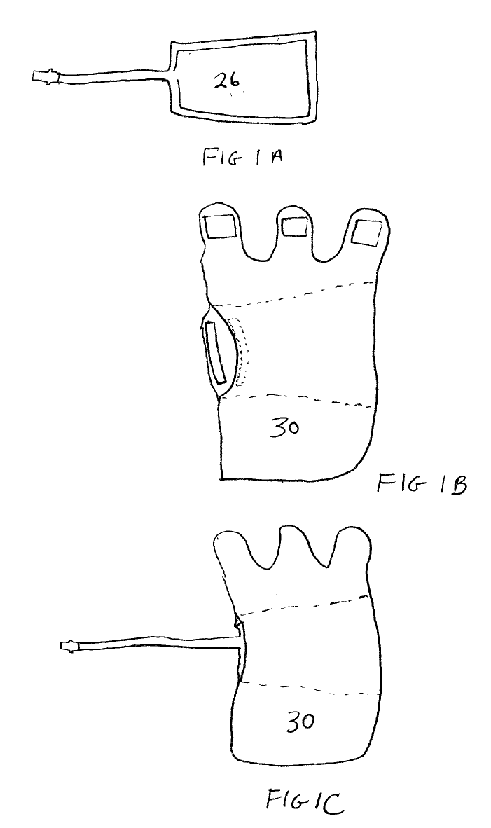

FIGS. 1A-1C show a typical bladder shape that may be covered and its bladder covering that is able to be placed and then removed from a shell that functions to wrap around a limb or an area.

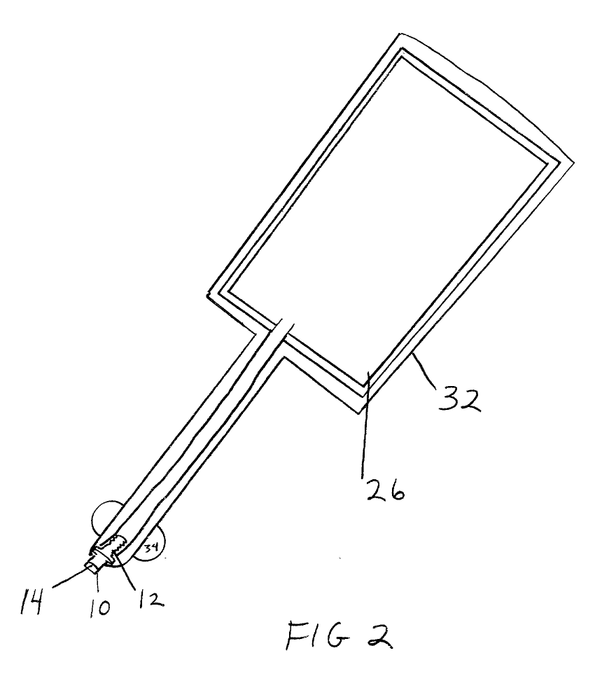

FIG. 2 shows the bladder and connector system covering to prevent contamination by the patient or the nurse

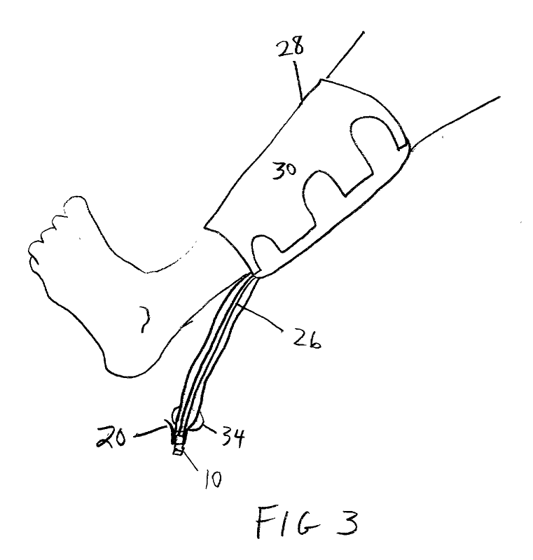

FIG. 3 shows an application of the protected bladder system in a generic external pneumatic compression device

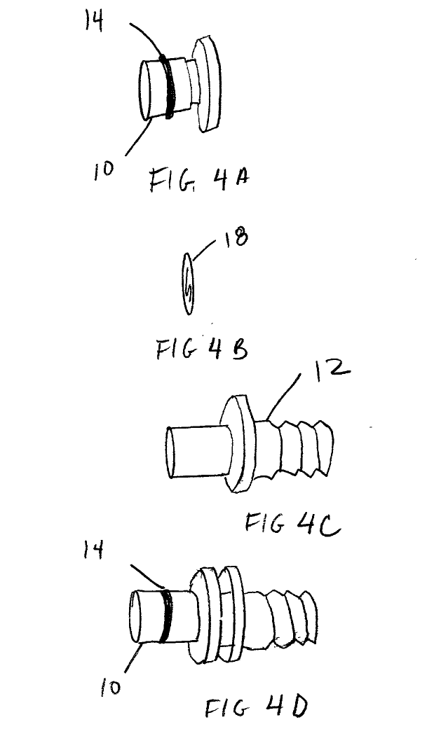

FIGS. 4A-4D shows the pneumatic connectors two main parts and the barrier membrane

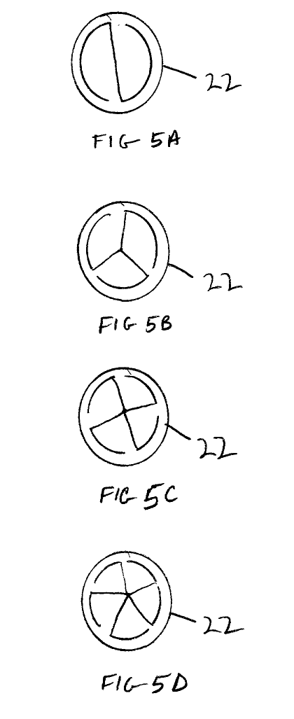

FIGS. 5A-5D shows the barrier membrane in various symmetrical configurations of design to give variable flow and contamination protection

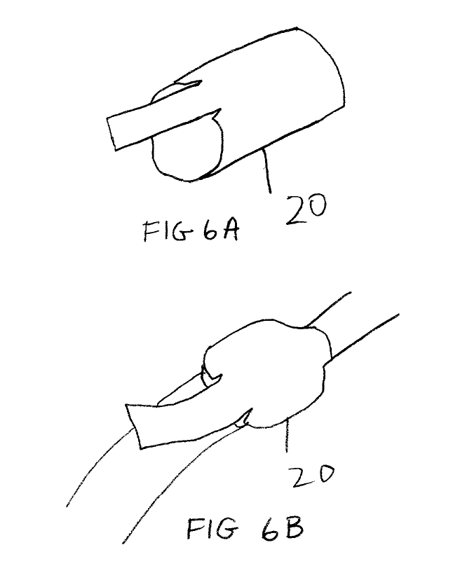

FIG. 6 shows the safety seal to connect and protect the bladder covering and the disposable portion of the pneumatic connector

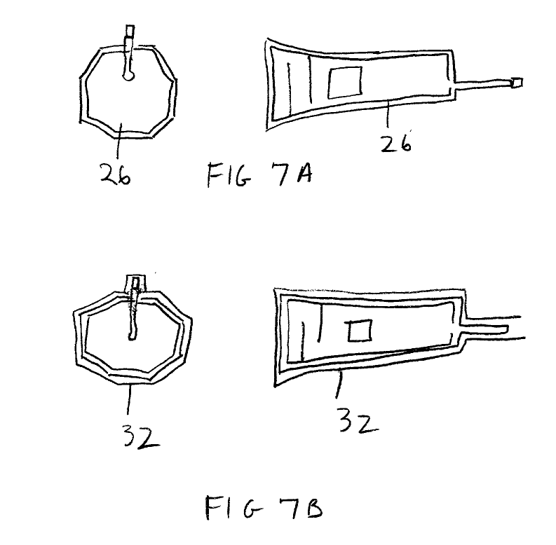

FIGS. 7A-7B shows two different shapes accommodated by different flexible covers to achieve a barrier protection of the inflation bladder, tube and coupler.

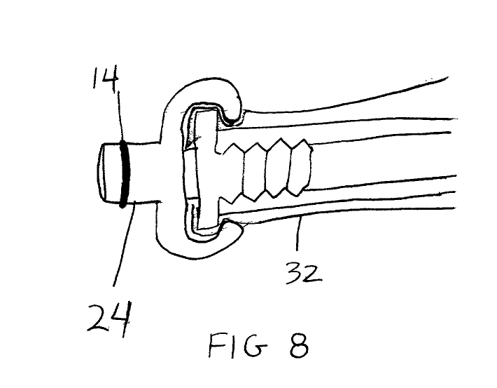

FIG. 8 shows two methods of attaching reusable coupler to the disposable coupler keeping the barrier intact without the need for the heat shrink device.

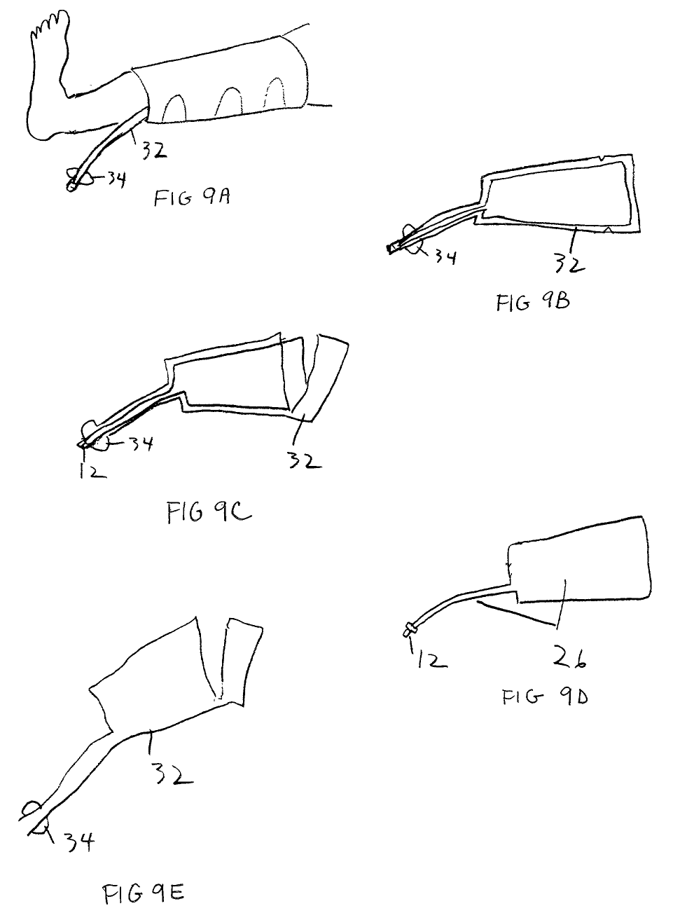

FIG. 9 shows the operation of the separable compression sleeve with barrier protection device and reusable coupler.

### REFERENCE NUMERALS IN DRAWINGS

| | |
|---|---|
| 10 outer or connector coupler shell | 12 reusable coupler portion |
| 14 sealing “o” ring | 16 seal heat shrink/connection |
| | groove |
| 18 membrane barrier with living hinge | 20 easy open heat shrink |
| | connection |
| 22 variable flow membranes | 24 disposable portion of |
| | coupler connector |
| 26 reusable bladder, tubing and connector | 28 disposable outer patient |
| | contact shell |
| 30 garment or sleeve | 32 protective flexible covering |
| | (barrier) |
| 34 ear on protective barrier | |

### DETAILED DESCRIPTION

Description—FIGS. 1A-1C—Preferred Embodiment

A preferred embodiment of the present invention is illustrated in FIG. 1A, 1B and 1C. The coupler portion 12 that is reusable is machined or molded such that its flow requirements are accommodated by the net internal diameter of the coupler and additionally the 22 flow membrane design which may use a mechanical plunger to force the hinge open upon connection or merely be designed with the appropriate number of symmetrical cuts and be molded or die cut of a thin enough memory retaining material such that the living hinge will open and allow passage of the required amount of fluid upon connection. The membrane 18 thickness, memory, durometer and resilience are chosen based on the application of the moving fluid and the associated pressure that and velocity in its required application. When the fluid is not actively moving the living hinge, the membrane shall have sufficient memory to return the membrane 18 to its original dimensions and planar shape to prevent aerosolized or tactile contamination. The reusable coupler length is proportioned in order that, when assembled with the disposable outer portion of the 10 coupler (containing the functional designs of sealing and connecting) the dimension remaining between the reusable portion of the coupler and coupler outer shell 10 where the membrane is positioned is less than the membrane thickness such that the compression of said membrane due to its entrapment between the two parts is sufficient to prevent the normal operational movement of fluid from removing it from its position. The 10 coupler or outer shell with said functional designs for sealing 14 and connecting 16 is machined or molded such that it fits over the reusable portion of the coupler with sufficient tolerance to prevent contamination and provide allowance for ease of assembly and disassembly after removal of the 20 shrink film connection.

The reusable bladder that contains the aforementioned couplers attached to the bladder (reusable and disposable(or sterilizable)) is surrounded by a protective flexible covering 32 with linear tear properties and barrier properties which prevents contamination from the outside or on patient contact. This covering has notches in the material at places where the linear tear of the covering will allow the exposure of the protected bladder so that it can be aseptically removed from the covering that protects it from external contamination.

The connection between the flexible polymer covering and the coupler outer shell 10 to both prevent contamination but allow for the ease of separation is the 20 heat shrink connection. This design is a one piece die cut of a heat shrink tube with linear tear properties along the length of the tube. After this heat shrink section has been shrunk around the connection of the outer flexible covering that covers the bladder and overlaps the disposable or sterilizable portion of the coupler, a notched tab will remain that when pulled will tear linearly and disconnect the tight seal that holds the flexible cover and disposable portion of the coupler together. This heat shrink seal also holds the diaphragm in the coupler. The tear of the coupler allows the flexible cover to pull over the reusable portion of the coupler attached to the tubing and not contaminate it with its outside surface. Small ears on the flexible cover are used to hold the cover when the bladder is ready to be pulled free of it. A notch is used on the outside of the flexible polymer covering to allow for tearing open the covering. Linear tear material is utilized to fabricate the flexible polymer covering so the tear at the notch is linear and provides for easy removal of the bladder or fluid device that is to be reused from the flexible polymer covering. The shrink fit connection is torn off which allows the contaminated portion of the coupler to be either disposed of or re-sterilized or reprocessed for a second use. The bladder and connector are protected by the flexible polymer covering. By grasping the ears on the covering near the coupler end and tearing the flexible bladder covering at the notch allows the bladder to be exposed for an aseptic removal and subsequent replacement into another flexible polymer covering which then will have added an outer connector with a diaphragm which then becomes secured together with the application and shrinking of the heat shrink connector. This covered bladder may then be placed inside a sleeve or garment where it can be placed against the desired substrate for compression with confidence that the bladder, connection tubing and connector (reusable portion) are protected from tactile contamination.

FIGS. 7A-7B—Additional Embodiments

Additional embodiments are shown in FIGS. 7A and 7B which show the bladder used in various devices where the covered bladder with two stage connector with diaphragm are utilized inside garments or sleeves to compress different portions of any body or device that needs compression providing a completely covered surface around all of the bladder including the inside of the tubing and bladder via the diaphragm.

Alternative Embodiments

There are various possibilities with regard to the relative shape of the covering in conjunction with the bladder. An additional embodiment would utilize an accordion flexible covering to cover stretchable pneumatic tubing. If a desired level of cleanliness was needed to be maintained inside the tube, additional levels of protection via a diaphragm or diaphragms (multiple) could be employed utilizing the same outer coupler sliding over a protected inner coupler with the heat shrink connection providing the compressive strength that holds the diaphragm inside the coupler to protect the inside from contamination.

Although this design includes the heat shrink connection to tightly secure and prevent liquid contamination of the junction of the reusable and disposable portion of the connectors, any design such as threaded or snap fit connections of the two connector pieces that would secure the flexible covering around the bladder or tubing in a connection that would prevent for example simple tactile contamination. FIG. 8 shows a design that would accomplish connection without the use of the heat shrink connection.

Advantages

From the description above, a number of advantages of the separable garment bladder system become evident:

(a) The basic design of having the entire pneumatic system (bladder, tubing, connector) separable from the garment or sleeve portion that contacts, wraps around and secures itself to the limb or device to be compressed allows different materials to be reprocessed in different modes. For example, there are methods to wash and clean garments that could be performed on the garment materiel that would not clean on a PVC bladder and tube for example, since liquids may be impossible to remove from a bladder yet could be easily dried in a garment or sleeve. Additionally, the bladder portion which in systems currently is disposable and therefore produces medical wastes (dioxins for example in the incineration of PVC) can now have choices to handle its reuse based on the need for a level of cleanliness in the device. A bladder can be simply cleaned externally or even reused if no certain level of microbial bioburden control is required. However, if a level of control is required, the bladder, tubing and connector may be covered with the barrier to insure tactile or liquid exposure with contamination does not contaminate the actual bladder material and the reusable portion of the connector.

(b) The covering of a device with notches placed and manufacturing of a linear tear material in the said flexible covering insures that an aseptic removal of the reusable bladder can be accomplished. When tubing is involved attached to a bladder, ears on the flexible covering assist in making certain the original bladder or tubing does not touch the contaminated outside of the flexible covering.

(c) The coupler portion is often the most expensive and most difficult portion of a reuse device to be cleaned especially when grooves are employed for “o” rings. By having a reusable portion being protected by a covering and an outer shell that provides merely the connection ability and the feature to secure additional levels of contamination prevention in the form of a diaphragm, the materials and labor expense is saved by not having to replace a complete connector (press fit it back into the tubing for example).

(d) The heat shrink connection device provides an inexpensive method to not only provide a secure liquid tight seal against contamination with its unique lack of linear placed perforations, the use of a linear tear shrink material with the notching provides a secondary function of holding the diaphragm in place through mechanical compression as well as providing a quick disassembly by a quick tear of the tab to separate the contaminated outer flexible covering for disposal from the inner protected bladder, tubing and connector.

(e) The design of a bladder overall in many cases is overkill as it must handle high pressures but its time of use is short and often of a disposable nature. The design of these features to provide a protected bladder and enable reuse without the limitations of toxic residues or destruction from standard sterilization procedures results in saving the worlds resources and getting the most out of a petroleum based product and additionally minimizes the exposure on the planet to otherwise premature incinerative by-products that result from not maximizing the usability of a product.

Operation—FIG. 9

The manner of using the separable compression sleeve with the barrier protected device and reusable coupler to allow the reuse of either a garment or sleeve portion or a bladder/tube and fluid coupler in a compressible sleeve device is as follows: After the use of the device for compression, the garment or sleeve portion pocket that secures the bladder is opened up and the bladder/tube/fluid coupler portion is removed. This feature allows the choice of handling the reprocessing of typically two different materials in separate methods. The garment/sleeve portion is often of a fabric material and washer dryer systems may be employed to handle this or the choice may be made to destroy this portion of the compressible sleeve. If there is no requirement for a level of cleanliness or a limit on the level of contamination, choices can be made on the type of reprocessing for the bladder/tube/fluid coupler.

If the reuse of the bladder/tube/coupler is desired and prevention of contamination is required, the flexible bladder covering, reusable and disposable fluid coupler with a diaphragm secured with a heat shrink connection may be employed. After removal of this covered bladder from the compressible limb garment, the heat shrink connection tab is pulled thereby tearing it linearly and removing the compressed connection of the bladder/tube covering from the reusable and disposable coupler. This is the first step in the removal of the bladder (non-contaminated) from the flexible outer covering (contaminated). The flexible outer covering around the bladder at its largest dimensional size will be notched so that it can be torn open.

The linear tear of this material allows the bladder to be exposed so that it can be aseptically removed from the contaminated outer covering by holding the ears placed typically near the end or the smallest dimension on the covering. The bladder is grabbed by non contaminated bands and pulled from the covering. In this manner no part of the outside of the bladder touches the outside of the flexible polymer covering. The disposable outer part of the coupler and diaphragm that was compressed between the coupler halves can be disposed of since it is contaminated or reevaluated for reuse after a form of reprocessing to restore it to a non contaminated condition where it may become a new barrier prevention on another compressible sleeve device.

Conclusion, Ramifications, and Scope

Accordingly, the reader will see that the separable compression sleeve with a barrier protected device and reusable coupler will allow new design levels of separable materials in compressible sleeves that allow for the reuse and reprocessing without the use of the standard methods of toxic gas or toxic chemical treatments that produce irremovable toxic residuals or penetrative radiation that tends to be destructive to the currently used materials and limit their reuse. Furthermore, the separable compression sleeve with a barrier protected device and reusable coupler has the additional advantages in that

It provides an easy method of separating bladder/tubing/coupler assemblies from the fabric sleeve portion that is used to wrap and hold the bladder around the limb or device to be compressed.

It allows for the multiple use of products that are typically disposable because reprocessing in standard methods still limits the multiple use due to toxic residues or degradation.

It allows for the reuse of fluid coupling devices without the need for sterilization or chemical processing by its barrier prevention design.

It provides a unique membrane which opens and control flow during use but when not in use provides a barrier against aerosolized or liquid spill contamination.

It provides an efficient way of securing the diaphragm and sealing from liquid contamination the outside flexible barrier cover over the bladder and the disposable fluid coupler portion of the coupler.

It provides a membrane which can be opened either mechanically via a male spear on the fluid coupler device or merely by fluid flow with a living hinge which due to its memory closes off the movement of mass through the fluid coupler when the coupler is separated.

It provides a process whereby in a compressible sleeve the bladder/tube/fluid coupler can be separated from the circumferential restraining device (sleeve) that contains the bladder/tube/fluid coupler and holds it in place around a circumference and allows it to be protected by a flexible outer covering over the surface of the bladder, tubing and reusable portion of the coupler, be barrier protected by a unique diaphragm valve that opens and closes during use but in non use stays closed to protect against contamination, and be held together by a heat shrink connection device that seals the bladder/tube/coupler from contamination and holds the diaphragm valve in place and can be easily removed by the quick pull of a tab so that the covering, diaphragm valve and connection portion of the fluid coupler can be quickly and aseptically removed and the non-contaminated bladder reused.

Although the description above contains many specificities, these should not be construed as limiting the scope of the invention but as merely providing illustrations of some of the presently preferred embodiments of this invention. For example, the diaphragm can have single or multiple placements to provide varied levels of barrier protection and have unique but symmetrical cuts to provide different level of fluid flow, etc.

Thus the scope of the invention should be determined by the appended claims and their legal equivalents, rather than by the examples given.

I claim:

1. In a compression sleeve that wraps circumferentially around a limb or desired substrate So as to position a bladder for compression via fluid entry and exit of said bladder a separable design where the bladder, tube, and fluid coupler portion can be separated from the sleeve so reprocessing procedures on their reuse can be executed.

2. The reuse of claim 1 whereby the bladder, tube, and coupler are protected from contamination by a barrier protection device consisting of a disposable flexible cover that completely encapsulates the bladder and tubing and a disposable or reprocessable coupler to function as a barrier to contamination of the reusable coupler connected by a unique heat shrink closure.

3. Said coupler of claim 2 is comprised of a reusable portion which is protected by the flexible barrier, a disposable or reprocessable coupler which attaches to the reusable portion of the coupler via a heat shrink closure to secure the flexible barrier cover and secure a unique diaphragm valve.

4. The unique diaphragm valve of claim 3 is a molded or die cut shape and based on the symmetrical shape of its die cut allows a specified flow based the number of symmetrical shapes that are symmetrically cut about the center of the disc by utilizing a living hinge and the material's inherent memory to return it to its original shape when fluid is not moving through it which creates a barrier to contamination.

5. The heat shrink closure of claim 2 has an extended tab that facilitates grabbing and has no perforations but is constructed of a linear tearing material so that based on the die cut of the shape pulling on the tab breaks the heat shrink closure from its hold on the reusable connector to the disposable or sterilizable connector so the bladder, tube, and coupler may be aseptically removed from the flexible cover and disposable coupler.

6. The heat shrink closure of claim 2 additionally holds in place by its shrink compression the diaphragm valve that selectively opens with flow and closes when no flow pressure is against it.
